(19) United States
(12) Patent Application Publication
(10) Pub. No.: US 2012/0238896 A1
(43) Pub. Date: Sep. 20, 2012

(54) METHOD FOR ELECTROIMPEDANCE TOMOGRAPHY

(75) Inventors: Yvo Gärber, Kastorf (DE); Thomas Gallus, Lubeck (DE)

(73) Assignee: Drager Medical GmbH, Lubeck (DE)

(21) Appl. No.: 13/365,512

(22) Filed: Feb. 3, 2012

(30) Foreign Application Priority Data
Mar. 16, 2011 (DE) 10 2011 014 107.3

(51) Int. Cl. A61B 5/053

(52) U.S. Cl. 600/547

(57) ABSTRACT

A method for electroimpedance tomography makes possible an analysis and reconstruction of an image of the electric resistance in the presence of a defect electrode (A). The method includes the steps of identifying an electrode as a defect electrode (A), which has no contact with the body, by means of an impedance measurement, of performing current feeds such that at least the defect electrode (A) is jumped over, and of determining voltage potentials in the area of the defect electrode (A) over the defect electrode (A) in such a way that the defect electrode (A) is jumped over at least once.

### CROSS REFERENCE TO RELATED APPLICATIONS

This application claims the benefit of priority under 35 U.S.C. §119 of German Patent Application DE 10 2011 014 107.3 filed Mar. 16, 2011, the entire contents of which are incorporated herein by reference.

### FIELD OF THE INVENTION

The present invention pertains to a method for electroimpedance tomography.

### BACKGROUND OF THE INVENTION

Electroimpedance tomography (EIT) is increasingly used in medicine. Typical EIT devices use 8, 16 or 32 electrodes for data acquisition, current being fed through two electrodes and the resulting voltage being measured between the remaining electrodes. By combining different feeds and measurements, it is possible to generate a signal vector, from which the impedance distribution can be determined by means of a suitable algorithm or the relative change in the impedance distribution relative to a reference value can be determined in the electrode plane in functional EIT (fEIT). The latter method is used in status-dependent functional electroimpedance of the thorax, in which N electrodes are arranged around the thorax in a ring-shaped pattern in order to reconstruct a tomogram of the ventilation-related relative impedance change, which is an indicator of the regional distribution of the ventilation of the lungs, from the comparison of the signal vectors in different states of the lungs, e.g., end-inspiratory and end-expiratory states. Thoracic fEIT is well suited for the regionally resolved lung monitoring of ventilation, especially in intensive care units in hospitals. A device for electroimpedance tomography is disclosed, for example, in U.S. Pat. No. 5,919,142 A.

The so-called adjacent data acquisition, in which current is fed through two adjacent electrodes (current carrying electrodes) and the voltages between the remaining electrodes is measured adjacent to each other, wherein current-carrying electrodes are left out because of the unknown voltage drop over the current-carrying electrodes, is a frequently used data acquisition strategy. Thus, thirteen voltage values are obtained for a current feed position. Thirteen voltages are again obtained for the current feed via a subsequent electrode pair, so that a total of 16*13=208 voltage measured values are present, from which the impedance distribution or the relative change in the impedance distribution can be determined with the use of 208 reference voltages with a reconstruction rule, which is applicable to this form of data acquisition. Such a data set, which contains all independent measurements without repetition at least once and is used to reconstruct an EIT image, is called a “frame.” A data set for a partial area is a “partial frame.” There also are numerous other data acquisition modes with current feed and/or voltage measurement over a plurality of electrodes, which is equivalent on the basis of reciprocity. The advantage of the adjacent data acquisition mode is the complete data space, because there are no more independent measured values. All other data acquisition modes can be reconstructed from the data space of the adjacent data acquisition mode in a simple manner on the basis of the linearity of the so-called Neumann-to-Dirichlet operator Λ_{σ}(I)→U; it can be easily imaged on an EIT hardware and has high sensitivity for determining relative changes in impedance.

There are various reconstruction methods for inferring the impedance distribution of the area enclosed by the electrodes from the measured voltages. Examples of reconstruction methods are the back projection method, Kalman filter-based techniques or sensitivity-based Newton-Raphson methods based on finite element models. The latter are frequently used nowadays because of the greater flexibility.

There is one thing all current EIT systems have in common in data acquisition and reconstruction. They operate only with the analysis of the data of the full electrode set. However, the case in which electric contact of an electrode or of a plurality of electrodes with the skin is not possible, especially when an easy-to-handle electrode belt is used, where the electrode position cannot be changed as desired, for example, because of dressings or drains, may not infrequently occur in clinical practice. Such contract-free electrodes will hereinafter be called defect electrodes. The current EIT systems fail in these cases. The system passes over into undefined states in the worst case, into a defined state in the best case, and data that can be processed further are obtained only if the defect electrode(s) has (have) contact again. The current EIT systems never deliver analyzable data in the disconnected case, because neither data acquisition nor reconstruction are designed for the failure of electrodes.

### SUMMARY OF THE INVENTION

A basic object of the present invention is to provide a method for electroimpedance tomography in order to make possible an analysis and reconstruction in the presence of at least one defect electrode.

The object is accomplished with the features of the method according to the present invention, which comprises the steps of identifying at least one electrode, which has no contact with the body, as a defect electrode by means of an impedance measurement, performing current feeds such that at least the defect electrode (A) is jumped over, and determining voltage potentials in the area of the defective electrode (A) over the defect electrode (A) in such a way that the defect electrode (A) is jumped over at least once.

According to another aspect of the invention, a device and system are provided.

The control and reconstruction software of the device is designed such that despite the absence or non-use of at least one electrode, measured data are obtained by jumping over this defect electrode or a plurality of defect electrodes. Impedance distributions or relative impedance distributions, which differ “non-substantially” from the results that would have been obtained during full functionality, are determined by means of a data analysis adapted to this operating state. “Non-substantially” means, for example, that the image dot difference of the (f)EIT image values between full and limited functionality does not differ by more than a preset value of image dot preset values, so that a medical interpretation is still possible. The EIT system is capable of identifying the defect electrode(s) itself(themselves), to send a message to the user, and to adapt them to the data acquisition mode (DAQ mode) and the reconstruction if the error cannot be eliminated.

The device for electroimpedance tomography is designed such that the EIT system assumes a defined state in case of failure of an electrode or of a plurality of electrodes for current feed and optionally voltage measurement, and the ability of the individual electrodes to function is monitored continuously, preferably by an electrode-skin contact transition impedance measurement. An electrode is considered to be unable to function if, for example, the electrode-skin contact transition impedances are above a certain threshold Zₒᵤₜ and again able to function if they are below a certain threshold Zᵢₙ, with Zᵢₙ≦Zₒᵤₜ (hysteresis switch). The subscript “in” designates within a permissible impedance range and “out” designates outside the permissible impedance range.

If inability to function of one or more electrodes is determined, the software of the EIT system is designed such that the reconstruction rule is adapted to the changed data acquisition, so that the impedances reconstructed therewith or the impedance changes or relative impedance changes do not differ, aside from minor, resolution-related differences from the standard reconstruction, and the essential information of the EIT image is preserved.

If repeated ability to function of one or more defect electrodes is determined, the hardware of the EIT system is designed such that the electrode(s) in question is (are) again integrated by control into the normal data acquisition by current feed and voltage measurement according to the standard DAQ mode used. If repeated ability of one or more defect electrodes to function is determined, the software of the EIT system is designed such that the reconstruction rule corresponding to the standard DAQ mode with re-integrated electrode(s) is used to determine the impedances or impedance changes or relative impedance changes.

The advantage of the method according to the present invention is that usable EIT measurements can be carried out with such an EIT system with minimal loss of information even in case of a defect electrode. An EIT system or EIT device cannot measure without such a method at all, or if it can, an area-dependent loss of sensitivity is associated with the loss of the measurements, so that the reconstruction contains a fuzzy to blind area in the image depending on the underlying basic DAQ mode. The disturbance is greatest for the adjacent DAQ mode because of the high sensitivity and maximum data space; the disturbances should be smaller depending on the spread in DAQ modes with electrodes inbetween, which will hereinafter be called “spread,” because jumpovers are provided even in the standards. The disturbances may, in turn, cover wider spaces based on the typically poorer basic sensitivity and resolution with increasing spread. However, depending on the spread, even such DAQ modes benefit from the jumpover principle, because, as soon as the defect electrode is reached, information is regained due to the jumpover, instead of having to accept the loss of this measurement.

The data space is maximally utilized in the EIT device according to the present invention due to the jumpover current feed/voltage measurement, and the loss of information in the EIT image is minimized by a correspondingly adapted reconstruction, so that the EIT image continues to be able to be interpreted.

In terms of contents, the N-electrode EIT system changes over into an N-D electrode EIT system, where N is the total number of electrodes used and D is the number of defect electrodes. This is a fundamental difference from eliminating the current feed and voltage measurements at the defect electrodes involved without jumping over, because the data space of N-D electrodes is not sufficiently covered here, wherein the data space of N-D electrodes has maximum coverage in case of jumpover.

It is likewise a fundamental difference from a plurality of possible DAQ modes and corresponding reconstruction rules (DAQ/REC), be it with current feed patterns with electrodes located in between and/or voltage measurements with electrodes located in between, because it is common to all that each electrode of the N electrode system is addressed. The corresponding reconstruction rules are always based on N electrodes, type DAQ/REC(N) for short, whereas the defect electrodes are entirely left out in the method according to the present invention.

An example of a 16-electrode EIT system with corresponding data acquisition is explained in the drawings. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing the principle of an impedance measurement with three electrodes;

FIG. 2 is a schematic view showing a data acquisition mode;

FIG. 3 is a schematic view showing an analysis method with a defect electrode;

FIG. 4a is a schematic view showing an example of current feeds to electrodes;

FIG. 4b is a schematic view showing an example of current feeds with a defect electrode;

FIG. 4c is a schematic view showing an example of current feeds with a defect electrode;

FIG. 5a is a view of a reconstruction corresponding to FIG. 4a;

FIG. 5b is a view of a reconstruction corresponding to FIG. 4b; and

FIG. 5c is a view of a reconstruction corresponding to FIG. 4c.

### DESCRIPTION OF THE PREFERRED EMBODIMENTS

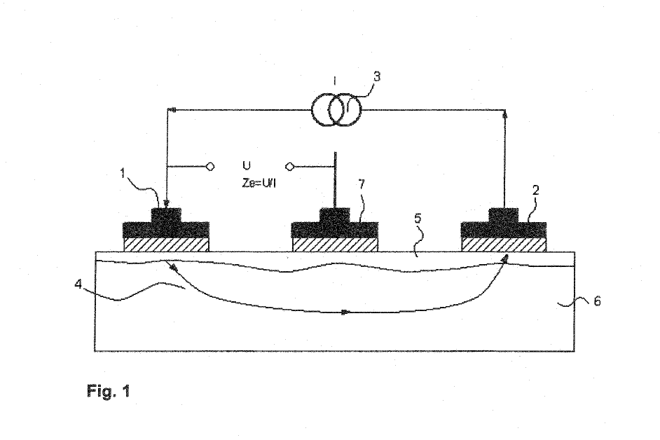

Referring to the drawings in particular, FIG. 1 schematically shows the principle of a three-point electrode-skin contact impedance measurement. The current I from a power source 3 is fed via two electrodes 1, 2. The current flows into the body 4 over an electrode 1 on the left and out again via an electrode 2 on the right. The body consists of upper skin layers 5 for contacting the electrodes 1, 2 and deeper skin and tissue layers 6. The voltage is measured from a current-carrying electrode against a currentless reference electrode. The main voltage drop at the current-carrying electrode 1 takes place at the transition to the interior of the body. The impedance is comparatively low in the body itself. The potential drop is measured against a currentless electrode 7, because there is no voltage drop here at the electrode-skin contact because I=0. The impedance Zₑ=U/I between the electrodes 1, 7 consequently represents essentially the electrode-skin contact transition impedance of the current-carrying electrode 1 being considered.

The electrode-skin contact transition impedances of all electrodes can thus be measured at least quasi-continuously, there typically being one measurement per partial frame. If an electric contact is not possible, the impedance increases greatly because of I→0.

FIG. 2 shows an example of a data acquisition in the adjacent DAQ mode for a 16-electrode EIT system. Partial frame 1: Current feed by means of power source 3 between the electrode pair α=1. All voltages between the electrode pairs μ=3 . . . 15 are measured, indicated by the example μ=6 and the lower rotation arrow 8. The electrode pairs with current-carrying electrodes are not measured, because the electrode-skin contact transition impedances are either unknown or too inaccurate because of fluctuations. Consequently, 13 voltage measured values are obtained for current feed position α=1. This is repeated for the current feed positions and partial frames α=2, α=3 . . . , α=16, indicated for the current arrow 9. The 13 voltages between the remaining adjacent electrodes are measured for each new current position. We obtain 16*13=208 measured values or 104 linearly independent measured values based on the reciprocity in the transposition of the feed site and measurement site. The subscripts are used as listed below. The concrete embodiment of this mode depends on the underlying hardware.

U_{α(μ)}=U_{σ}(I_{μ})

μ,α=1 . . . , 16 electrodes

α(μ)→m ∈[1, . . . , 208]_{channel}

m=1 corresponds to (μ=1, α=3)

m=2 corresponds to (μ=1, α=4)

m=208 corresponds to (μ=16, α=14)

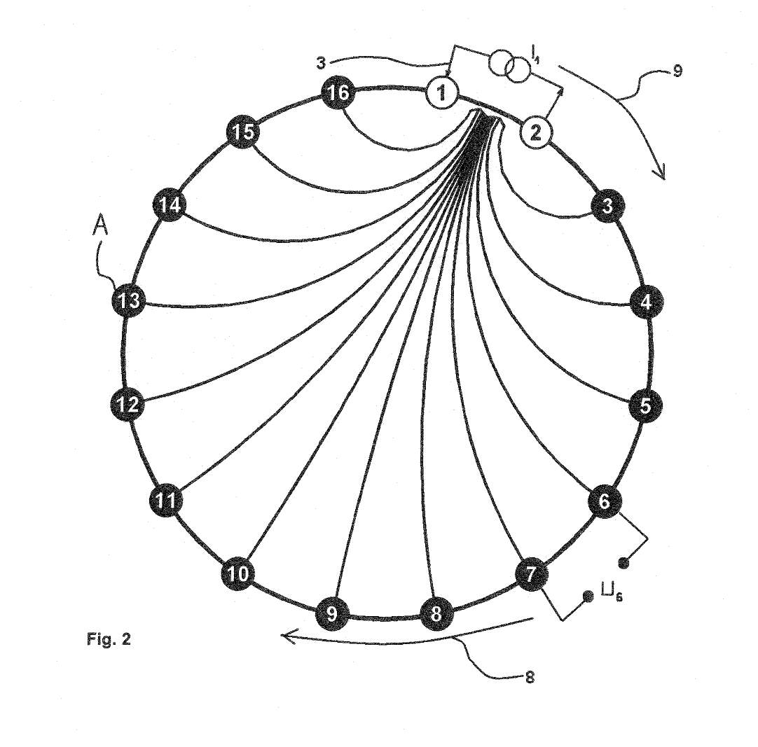

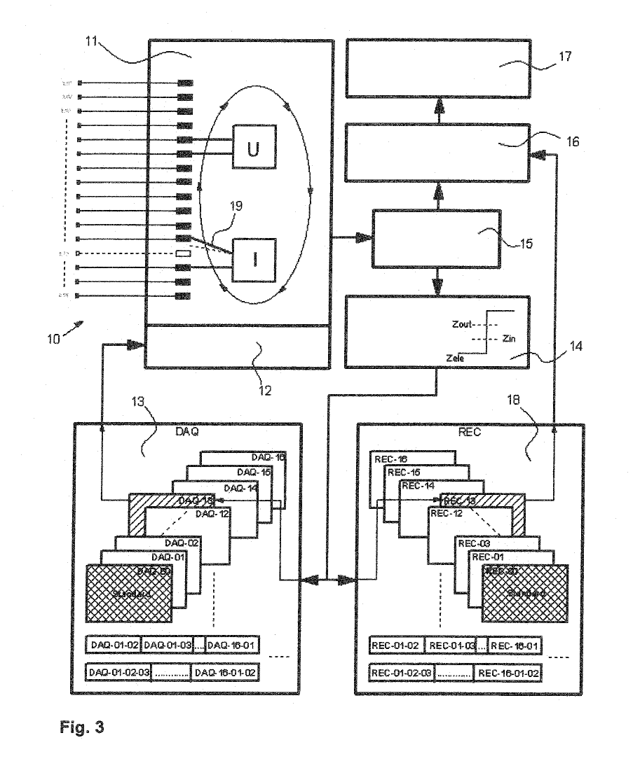

FIG. 3 schematically shows the measuring method on the basis of a block diagram 10 based on the example of a 16-electrode EIT system with adjacent DAQ mode and failure of an electrode A corresponding to FIG. 2.

The 16 electrodes are connected to a DAQ circuit 11 with DAQ control 12 on a DAQ pattern basis 13.

The defect electrode is identified by the exceeding or undershooting of threshold values for the impedance Zₒᵤₜ or impedance Zᵢₙ, where Zₙ is typically lower than Zₒᵤₜ (hysteresis threshold). The analysis is performed by means of an impedance monitoring unit 14. Current feed pattern and voltage measurements are performed by means of the DAQ hardware. For example, cascade-like multiplexer circuits offer the possibility of embodying pairs for current feed and voltage measurement corresponding to the preset DAQ pattern.

The 208 voltage measured values and the 16 measured values of the electrode-skin contact transition impedances are read and are typically sent to an A/D converter 15 and are subjected to preprocessing. The voltage measured values are sent to a computing unit 16 for reconstruction and image processing and are further processed on the basis of a reconstruction rule, REC rule, from a data bank 18, and outputted via a display unit 17.

The 16 electrode-skin contact transition impedances are sent to the impedance monitoring unit 14. Electrode A=13 is identified in this example on the basis of excessively high impedance values above the threshold Zₒᵤₜ as a defect electrode. The system assumes a defined safe state.

The information is sent and a data bank into the different DAQ patterns for the standard case without defect electrode as well as for (DAQ-00) and also for the 16 different defect electrodes DAQ-01 . . . DAQ-16. Even more patterns may be possibly stored for possibly more defect electrodes. The jumpover pattern DAQ-13 for defect electrode A is loaded into the DAQ control unit. The DAQ unit controls the electrodes now thus that electrode A is jumped over by the current feed and voltage in a defined manner, indicated by the open switch with the broken line 19 in FIG. 3. The jumpover patterns may vary depending on the hardware possibilities. The DAQ now begins with the data recording corresponding to the new DAQ pattern for defect electrode A. The data are read, subjected to A/D conversion, and go to the computing unit 16 and impedance monitoring unit 14.

The information of the defect electrode A from the impedance monitoring unit 14 is likewise sent to the data bank 18 for the reconstruction rules corresponding to the corresponding DAQ modes, which were calculated in advance. It contains the standard reconstruction rule without defect electrode (REC-00), that for the 16 different possibilities for a defect electrode (REC-01 . . . REC-16) and possibly even more rules for larger numbers of defect electrodes. The different reconstruction rules may, of course, also be calculated completely or partly at the site, depending on how the memory space and computing capacity are distributed.

The inquiry and data structure for DAQ modes and modes of reconstruction may likewise be different; what is important is that both must always be changed: DAQ and reconstruction rule.

The reconstruction rule REC-13 for the jumpover over the defect electrode A is loaded and sent to the reconstruction and image processing unit. The voltages measured [“gemessen” in line 28, p. 8 of original is a typo for “gemessenen”—Tr. Ed.] in the new DAQ mode, DAQ-13, can now be reconstructed, analyzed, displayed and possibly stored with minimal loss of information.

In the input impedance of electrode A drops again below a threshold value Zᵢₙ or other electrodes fail, this is noticed by the electrode impedance monitoring unit and responded to analogously in such a way that the best possible image quality can always be generated.

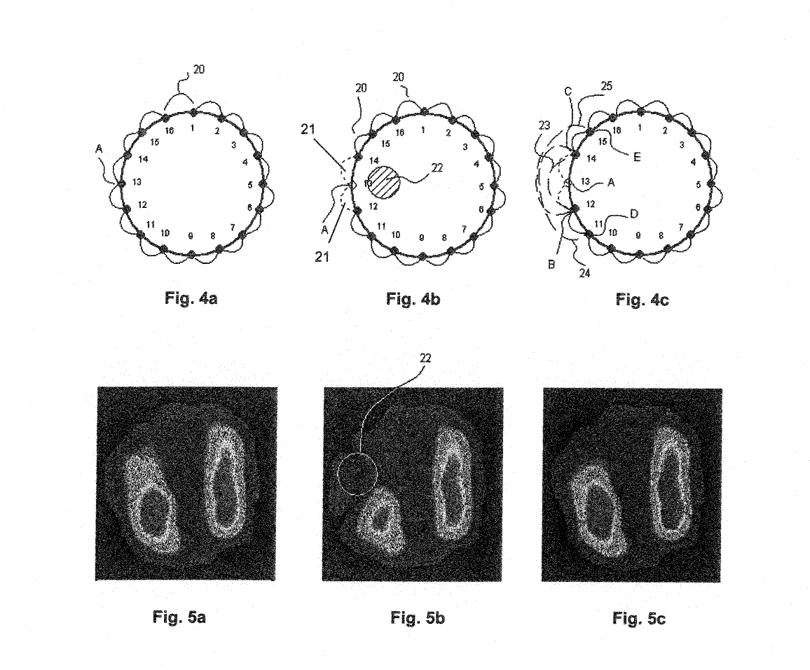

FIGS. 4a through 4b show different DAQ patterns for current feeds in the area of the defect electrode A.

FIG. 4a shows the arches 20 for undisturbed current feeds and voltage measurements, where all 16 electrodes have contact with the skin surface.

The defect electrode A has no contact in the case illustrated in FIG. 4b and it is simply omitted in the data acquisition. The arches 21 illustrate here the current feeds and voltage measurements left out.

The simple omission without jumping over leads to highly unsatisfactory results. For example, 52/208 measurements, i.e., ¼ of all data, i.e., two whole partial frames, and two measurements from every other partial frame, would be discarded in case of only one defect electrode. This causes that no information is present from the area close to the defect electrode A, which is illustrated as a “blind spot” 22 in FIG. 4b. This leads to a great disturbance in the EIT image in this area.

When jumping over the defect electrode A, as is illustrated in FIG. 4c, a large part of the information can be recovered from the area affected, albeit with a somewhat lower resolution, which leads to usable EIT images. When jumping over directly, 15*12=180 measurements are obtained with electrodes B, C located adjacent to one another with 15 electrodes, and 14*11=154 measurements are obtained in case of two defect electrodes with an adjacent mode. At least one jumpover 23 is necessary relative to the defect electrode A. Not only are more data available than when omitting, but above all data that are sensitive to a change in impedance in the area affected are available, which means a marked gain of information.

The reconstruction can be improved further by additional jumpovers 24, 25 over the defect electrode A. The jumpover 24 begins at the electrode D located in front of electrode B and goes to electrode C. The manner of concrete jumpover depends on the concrete hardware resolution, e.g., the concrete resolution of a multiplexer cascade.

The effects of the defect electrode A in the EIT image of the ventilation of the lung of a test subject are illustrated in FIGS. 5a through 5c. The data were recorded with a 16-electrode EIT system in the adjacent DAQ mode. FIG. 5a corresponds to FIG. 4a with complete data set of all 16 electrodes. FIG. 5b shows the effect of the blind spot 22 in case of the unacceptable omission of the measurements in question around the defect electrode A, corresponding to FIG. 4b. FIG. 5c illustrates the regain of information by jumpover measurement with a slight loss of resolution. It is seen in FIGS. 5a through 5c that the functionality and interpretability of EIT remains fully preserved with the jumpover method according to the present invention.

While specific embodiments of the invention have been shown and described in detail to illustrate the application of the principles of the invention, it will be understood that the invention may be embodied otherwise without departing from such principles.

APPENDIX
List of Reference Numbers

| | |
|---|---|
| 1 | Left electrode |
| 2 | Right electrode |
| 3 | Power source |
| 4 | Body |
| 5 | Upper skin layer |
| 6 | Deeper tissue layer |
| 7 | Currentless electrode |
| 8 | Lower rotation arrow |
| 9 | Current arrow |
| 10 | Block diagram |
| 11 | DAQ circuit |
| 12 | DAQ control |
| 13 | DAQ pattern basis |
| 14 | Impedance monitoring unit |
| 15 | A/D converter |
| 16 | Computing unit |
| 17 | Display unit |
| 18 | Data bank |
| 19 | Broken line |
| 20 | Arch for undisturbed measurement |
| 21 | Arch for omitted measurement |
| 22 | Blind spot |
| 23, 24, 25 | Jumpover |
| A | Defect electrode |
| B, C | Adjacent electrode to the defect |
| | electrode |
| D, E | Electrode located in front of the |
| | adjacent electrode |

What is claimed is:

1. A method for data acquisition by means of a device for electroimpedance tomography, the method comprising the steps of:
attaching electrodes conductively to a circumference of a test subject at spaced locations in order to generate an image of the electric resistance over the cross-sectional area covered by the electrodes by means of a reconstruction algorithm from voltage potential difference measurements caused at the remaining electrodes by current feeds via the remaining electrodes;
identifying an electrode as a defect electrode, which has no contact with the body, by means of an impedance measurement;
performing current feeds such that at least the defect electrode is jumped over; and
determining voltage potentials over the defect electrode in an area of the defect electrode in such a way the defect electrode is jumped over at least once.

2. A method in accordance with claim 1, wherein a first jumpover takes place over the electrodes located adjacent to the defect electrode.

3. A method in accordance with claim 2, wherein the electrodes located adjacent to the defect electrode are jumped over in addition to the defect electrode during a further jumpover.

4. A method in accordance with claim 1, wherein the electrodes located adjacent to the defect electrode are jumped over in addition to the defect electrode during a further jumpover.

5. A method in accordance with claim 3, wherein an adjacent electrode and another adjacent electrode, which is located in front of or after the adjacent electrode, is used in a second or third jumpover.

6. A method of electroimpedance tomography data acquisition comprising the steps of:
attaching electrodes conductively to a circumference of a test subject at spaced locations;
generating an image of the electric resistance over the cross-sectional area covered by the electrodes using a reconstruction algorithm from voltage potential difference measurements between measuring pairs of electrodes upon current feed at a feed pair of electrodes wherein a feed position and measuring positions are changed to change the feed pair of electrodes and to change the measuring pairs of electrodes and a data set of voltage potential difference measurements between measuring pairs of electrodes is provided for the feed pair of electrodes and the electrodes comprising the feed pair of electrodes change for each feed position to provide changed measuring positions with further data sets of voltage potential difference measurements;
identifying an electrode as a defect electrode by impedance measurement;
performing current feeds such that an electrode adjacent to the defect electrode is used to form the feed pair of electrodes instead of the defect electrode;
determining voltage potential differences such that an electrode adjacent to the defect electrode is used to form one of the measuring pairs of electrodes instead of the defect electrode.

7. A method in accordance with claim 6, further comprising performing a current feed such that an electrode adjacent to the electrode adjacent to the defect electrode is further used to form an additional feed pair of electrodes instead of the defect electrode.

8. A method in accordance with claim 6, further comprising determining a voltage potential difference such that an electrode adjacent to the electrode adjacent to the defect electrode is further used to form one of the measuring pairs of electrodes instead of the defect electrode.

9. A method in accordance with claim 6, wherein said step of identifying an electrode as a defect electrode by impedance measurement includes monitoring an electrode-skin contact transition impedance measurement to determine if electrode-skin contact transition impedances are within a permissible impedance range.

10. An electroimpedance tomography data acquisition system comprising:
electrodes for attachment conductively to a circumference of a test subject at spaced locations;
a control unit for generating an image of the electric resistance over the cross-sectional area covered by the electrodes using a reconstruction algorithm from voltage potential difference measurements between measuring pairs of electrodes upon current feed at a feed pair of electrodes wherein a feed position and measuring positions are changed to change the feed pair of electrodes and to change the measuring pairs of electrodes and a data set of voltage potential difference measurements between measuring pairs of electrodes is provided for the feed pair of electrodes and the electrodes comprising the feed pair of electrodes change for each feed position to provide changed measuring positions with further data sets of voltage potential difference measurements, the control unit:
identifying an electrode as a defect electrode by impedance measurement;
performing current feeds such that an electrode adjacent to the defect electrode is used to form the feed pair of electrodes instead of the defect electrode;
determining voltage potential differences such that an electrode adjacent to the defect electrode is used to form one of the measuring pairs of electrodes instead of the defect electrode.

11. A system in accordance with claim 10, wherein the control unit further performs a current feed such that an electrode adjacent to the electrode adjacent to the defect electrode is further used to form an additional feed pair of electrodes instead of the defect electrode.

12. A system in accordance with claim 10, wherein the control unit further determines a voltage potential difference such that an electrode adjacent to the electrode adjacent to the defect electrode is further used to form one of the measuring pairs of electrodes instead of the defect electrode.

13. A system in accordance with claim 6, wherein the control unit identifies an electrode as a defect electrode by impedance measurement by monitoring an electrode-skin contact transition impedance measurement to determine if electrode-skin contact transition impedances are within a permissible impedance range.
